(19) United States
(12) Patent Application Publication
(10) Pub. No.: US 2006/0141596 A1
(43) Pub. Date: Jun. 29, 2006

(54) Process for the immobilization of enzymes

(76) Inventors: Sumant Phadtare, Maharashtra (IN); Murali Sastry, Maharashtra (IN)

Correspondence Address: OSTROLENK, FABER, GERB & SOFFEN, 1180 Avenue of the Americas, New York, NY, 10036-8403, US

(21) Appl. No.: 11/023,903

(22) Filed: Dec. 28, 2004

(51) Int. Cl. C12N 11/14

(52) U.S. Cl. 435/176; 977/702

(57) ABSTRACT

The present invention relates to a novel process for the immobilization of the enzyme on biocompatible gold nanoparticle encapsulated free-standing membranes. The binding of the enzyme occurs through the amine groups and cysteine residues to the gold nanoparticles. The free-standing gold nanoparticle membrane was synthesized at the interface between chloroform containing bis(2-(4-aminophenoxy)ethyl)ether (DAEE) and aqueous chloroauric acid solution. The membrane is formed spontaneously by the reduction of AuCl₄⁻ ions by DAEE, this process leading to the formation of gold nanoparticles. The concomitant process of oxidation of DAEE leads to the creation of a polymeric matrix in which the gold nanoparticles are embedded. The gold nanoparticle membrane is extremely stable, robust, easily handled, malleable and can be grown over large areas and thickness by suitably varying the experimental conditions.

### FIELD OF THE INVENTION

The present invention relates to a novel process for the immobilization of enzymes. More particularly the present invention relates to a process of immobilization using biocompatible gold nanoparticle encapsulated free-standing membranes. The present invention also provides for bioconjugation of an enzyme to gold nanoparticles in a free-standing membrane through amine groups and cysteine residues present in the enzymes. The binding of the enzyme is accomplished by immersion of the nanogold free-standing membrane in the enzyme solution with mild stirring. The fee-standing gold nanoparticle membrane is synthesized at the interface between chloroform containing bis(2-(4-aminophenoxy)ethyl)ether (DAEE) and aqueous chloroauric acid solution. The membrane is formed spontaneously by the reduction of AuCl₄⁻ ions by DAEE, this process leading to the formation of gold nanoparticles. The concomitant process of oxidation of DAEE leads to the creation of a polymeric matrix in which the gold nanoparticles are embedded. The gold nanoparticle membrane is extremely stable, robust, easily handled, malleable and can be grown over large area and thickness by suitably varying the experimental conditions.

### BACKGROUND OF THE INVENTION

Immobilized enzymes are used in organic syntheses to fully exploit technical and economical advantages of biocatalysts based on isolated enzymes. Immobilization enables separation of enzyme catalyst easily from reaction mixture, and can lower costs of enzymes dramatically. This is true for immobilized enzyme preparations that provide a well balanced overall performance, based on reasonable immobilization yields, low mass transfer limitations, and high operational stability.

Several methods are available for immobilization, which span from binding on prefabricated carrier materials to incorporation into in-situ prepared carriers. Operative binding forces vary between weak multiple adsorptive interactions and single attachments through strong covalent binding. The appropriateness of the method is usually a matter of the desired application

In addition to a more convenient handling of enzyme preparations, the two main targeted benefits are (1) easy separation of the enzyme from the product, and (2) reuse of the enzyme. Easy separation of enzyme from product simplifies enzyme applications and supports a reliable and efficient reaction technology. On the other hand, reuse of enzymes provides cost advantages, which are often an essential prerequisite for establishing an enzyme-catalyzed process.

### DESCRIPTION OF THE PRIOR ART

Biotechnology is witnessing impressive advances in the synthesis of biocompatible surfaces for the immobilization of a range of biomolecules such as DNA, enzymes and whole cells with important applications in biosensing and medicine. Encapsulation also protects the enzymes against degradation, aggregation and deamidation while rendering the enzymes accessible to substrates and co-factors for biosensing and biocatalytic applications. An important requirement for immobilizing the proteins is that the matrix should provide a biocompatible and inert environment, i.e. it should not interfere with the native structure of the protein and thereby compromise its biological activity.

The combination of nanoscale inorganic materials with organic polymers has a high potential for future applications such as device technology and drug delivery and has therefore attracted, a lot of attention during the last decade. Polymer systems have played important roles as nano-templates with different morphologies and tunable sizes for nanofabrication of all kinds of inorganic materials, as they can be easily removed after reactions, and can be further modified with different functional groups to enhance their interactions.

Development of experimental processes for the synthesis of biocompatible surfaces for the immobilization of the range of biomolecules is important not only from a fundamental point of view but also in biosensing and medicine. In particular gold nanoparticles have proved to be a biocompatible surface for the immobilization enzymes. Gold and silver nanoparticles have been functionalized with the immunoglobulins of class G and E (IgG and IgE, respectively). Such nanoparticles had a specificity directed against either the D-biotin or the dinitriphenyl (DNP) groups (Shenton et al Advanced Materials 1999, 11, 449). Electrode immobilized layers of gold nanoparticles for the adsorption of enzyme horse-radish peroxidase to prepare biosensors for the electrocatalytic detection of hydrogen peroxide (Patolsky et al. J. Electroanal. Chem. 1999, 479, 69). Polymeric membrane that contains a collection of monodisperse gold nanotubules, with varying inside diameter are been used for the separation of small molecules on the basis of molecular size (Jirage et al Sience 1997, 278, 655). Such chemically functional polymeric gold nanotubule membranes have potential applications in chemical selective separation of compounds (Hulteen et al J. Am. Chem. Soc. 1998, 120, 6603).

U.S. Pat. No. 6,602,932 provides a process for nanoparticle composites and nanocapsules for guest encapsulation and methods for synthesizing same. One synthesis method includes providing a nanoparticle template; and forming a shell on the nanoparticle template by polymerizing a monomer on the nanoparticle template to form a nanoparticle composite defined by the shell and the nanoparticle template. Another synthesis method includes providing a nanoparticle template; forming a shell on the nanoparticle template by polymerizing a monomer on the nanoparticle template; and dissolving the nanoparticle template to thereby form a hollow nanocapsule defined by the shell. Another synthesis method includes providing a nanoparticle template carrying a guest molecule; and forming a shell on the nanoparticle template by polymerizing a monomer on the nanoparticle template to thereby encapsulate the guest molecule. This method can also include dissolving the nanoparticle template to form a nanocapsule defined by the capsule shell material, wherein the guest material resides in the nanocapsule.

U.S. Pat. No. 6,486,334 present an invention relates to artificial biocompatible surfaces. More particularly it relates to surfaces, which mimic monolayer phospholipid structures in humans. Disclosed are thiol-functionalized phospholipids that have been covalently linked to a gold and/or silver substrate, methods for making them, and intermediates useful for such purposes. The resulting material creates a biomimetic surface that can be included in a conduit containing blood.

The major drawbacks of the prior art process are:

1. The immobilized enzymes on the different membranes have poor reusability.

2. It is difficult to separate the enzyme from the reactions mixtures and hence, the separation of products is difficult and costly.

3. It is difficult and costly to separate the free enzyme from the reaction mixture for the reuse and is not economically viable to the industry.

4. The free enzyme in solution is not stable and does not show any biocatalytic activity for a longer period of time and hence is not used for the longer period.

5. After binding of the enzymes to different solid supports they tend to show decrease in biocatalytic activity as compared to free enzyme in solution.

6. It is difficult to immobilize more than one enzyme on a single solid substrate for the multi step reactions.

7. Generally enzymes immobilized on the different solid supports cannot withstand high temperature and pH conditions during the biocatalytic reaction as compared to free enzyme in solution.

### OBJECT OF THE INVENTION

The main object of the invention is to provide a novel process for synthesis of a biocompatible surface in the form of nanogold membrane for immobilization of enzymes.

Another object of the invention is to reuse an enzyme immobilized on the nanogold membrane.

### SUMMARY OF THE INVENTION

Accordingly the present invention provides a process for the preparation of immobilized enzymes on nano gold particles the method comprising mixing a dilute solution of chloroauric acid and a bis(2-(4-aminophenoxy)ethyl)ether (DAE) in solvent and keeping the mixture in dark for a period of at least three hours to obtain a nanogold membrane at the organic/aqueous liquid-liquid interface, removing the membrane from the mixture, washing with double distilled water, drying at room temperature, contacting the gold nano membrane with a solution in buffer of enzyme to be immobilized, washing the membrane with buffer and drying to obtain the immobilized enzymes on nano gold membrane.

In one embodiment of the invention the concentration of chloroauric acid is in the range of 10⁻² M to 10⁻⁴ M.

In another embodiment the concentration of bis(2-(4-aminophenoxyethyl)ether (DAEE) is in the range of 10⁻² M to 10⁻⁴ M.

In another embodiment the solvent for preparing solution of bis(2-(4-aminophenoxy)ethyl)ether (DAEE) is selected from the group consisting of chloroform, dichloromethane and ethyl acetate.

In another embodiment the enzyme to be immobilized is selected from the group consisting of pepsin, fungal-protease, trypsine, endoglucanase and penicillin G acylase.

In another embodiment the buffer used for the preparing solution of enzyme is selected from the group consisting of KCl-HCl, glycine-HCl, sodium citrate buffer, sodium phosphate buffer and glycine-NaOH buffer.

In another embodiment the concentration of enzyme is in the range of 10⁻²M to 10⁻⁹ M.

### DETAILED DESCRIPTION OF THE INVENTION

The present invention resides in a novel process for the immobilization of enzymes on the surface of biocompatible gold nanoparticle encapsulated free-standing membranes as a scaffold. The membranes are obtained by a dilute solution of chloroauric acid and a bis(2-(4-aminophenoxy)ethyl)ether (DAEE) in solvent, keeping the mixture in dark for a period of at least three hours, to obtain a nanogold membrane at the organic/aqueous liquid-liquid interface. The membrane is then removed from the mixture, washed with double distilled water, dried at room temperature. The gold nano membrane is contacted with a solution in buffer of the enzyme to be immobilized, and then washed with buffer and dried to obtain the immobilized enzymes on nano gold membrane.

The process for the preparation of immobilized enzymes on nano gold particles resides in first mixing a dilute solution of chloroauric acid and bis(2-(4-aminophenoxy)ethyl)ether (DAEE) in solvent. This mixture is kept in dark for a period of at least three hours to obtain a nanogold membrane at the organic/aqueous liquid-liquid interface. The membrane is then removed from the mixture and washed with double distilled water and then dried at room temperature. The dried gold nano membrane is contacted with a solution in buffer of enzyme to be immobilized and then washed with buffer and dried to obtain the immobilized enzymes on nano gold membrane. The concentration of chloroauric acid is in the range of 10⁻² M to 10⁻⁴ M while the concentration of bis(2-(4-aminophenoxy)ethyl)ether (DAEE) is in the range of 10⁻² M to 10⁻⁴ M.

The solvent for preparing solution of bis(2-(4-aminophenoxy)ethyl)ether (DAEE) is selected from the group consisting of chloroform, dichloromethane and ethyl acetate. The enzyme to be immobilized can be enzymes such as pepsin, fungal-protease, trypsine, endoglucanase and penicillin G acylase, etc. The concentration of enzyme is preferably in the range of 10⁻² M to 10⁻⁹ M. The buffer used for the preparing solution of enzyme is selected from the group consisting of KCl-HCl, glycine-HCl, sodium citrate buffer, sodium phosphate buffer and glycine-NaOH buffer.

It is observed that by simple immersion of the nanogold membrane in the enzyme solution (aqueous) results in the binding of the enzyme to the gold nanoparticles present in the polymeric membrane.

The process of the invention is described herein below with reference to the following illustrative examples, which should not be construed as limiting the scope of the invention.

### EXAMPLE 1

30 mL of 10⁻³ M chloroauric acid (HAuCl₄) and 30 mL of 10⁻³ M bis(2-(4-aminophenoxy)ethyl)ether (DAEE) in CHCl₃ were mixed in a beaker kept under static ambient conditions in the dark for three hours. After three hours of reaction, a purple membrane was observed to have formed at the organic/aqueous liquid-liquid interface. The nanogold membrane was removed from the liquid-liquid interface by lifting with forceps, washed thoroughly several times with double distilled water and dried before further use. Immobilization of enzyme pepsin was done by immersing as prepared nanogold membranes (10 mg) in 10 ml of 0.5 mg/mL of concentration of the enzyme solutions (pH 2, 0.02 M KCl-HCl buffer) for 1 h. After enzyme immobilization, the nanogold membranes were washed thoroughly several times with KCl-HCl buffer solution and were stored at 4° C. and were used further. The enzyme-nanogold membranes bioconjugate were used for biocatalytic measurements using casein as the substrate. The membranes were reused for at the least 10 times. Prior to reuse the membranes were washed with respective buffer solutions. Stability of the enzyme pepsin bound to the nanogold membrane bioconjugates was checked in temperatures range 20-60° C. and in 2-8 pH range.

### EXAMPLE 2

This example illustrates the immobilization of the enzyme fungal protease on the nanogold membrane. Immobilization of the enzyme fungal protease was done by immersing as-prepared nanogold membranes (10 mg) in 10 ml of 0.5 mg/mL of concentration of enzyme solutions (pH 3, 0.05 M glycine-HCl buffer) for 1 h. After enzyme immobilization, the nanogold membranes were washed thoroughly several times with glycine-HCl buffer solution and were stored at 4° C. and were used further. The enzyme-nanogold membrane bioconjugate were used for biocatalytic measurements using hemoglobin as the substrate. The membranes were reused for at the least 10 times. Prior to reuse the membranes were washed with respective buffer solutions. Stability of the enzyme pepsin bound to the nanogold membrane bioconjugates was checked in temperatures range 20-60° C. and in 2-8 pH range.

### EXAMPLE 3

This example illustrates immobilization of enzyme invertase on nanogold membrane. Immobilization of enzyme invertase was done by immersing as-prepared nanogold membranes (10 mg) in 10 ml of 0.5 mg/mL of concentration of the enzyme solutions (pH 4.5, 0.05 M sodium acetate buffer) for 1 K, After enzyme immobilization, the nanogold membrane was washed thoroughly several times with sodium acetate buffer solution and were stored at 4° C. and was used further. The enzyme-nanogold membranes bioconjugate were used for biocatalytic measurements using sucrose as the substrate. The membranes were reused for at the least 10 times. Prior to reuse membranes were washed with respective buffer solutions. Stability of the enzyme invertase bound to the nanogold membrane bioconjugates was checked in temperatures range 20-60° C. and in 2-8 pH range..

### EXAMPLE 4

This example illustrates immobilization of enzyme penicillin G acylase on nanogold membrane. Immobilization of enzyme penicillin G acylase was done by immersing as-prepared nanogold membranes (10 mg) in 10 ml of 0.5 mg/mL of concentration of enzyme solutions (pH 7.5, 0.05 M sodium phosphate buffer) for 1 h. After enzyme immobilization, the nanogold membranes were washed thoroughly several times with sodium acetate buffer solution and were stored at 4° C. and were used further. The enzyme-nanogold membranes bioconjugate were used for biocatalytic measurements using potassium salt of penicillin G as the substrate. The membranes were reused for at the least 10 times. Prior to reuse membranes were washed with respective buffer solutions. Stability of the enzyme penicillin G acylase bound to the nanogold membrane bioconjugates was checked in temperatures range 20-60° C. and in 2-8 pH range.

### EXAMPLE 5

This example illustrates immobilization of enzyme endoglucanase on nanogold membrane. Immobilization of enzyme endoglucanase was done by immersing as-prepared nanogold membranes (10 mg) in 10 ml of 0.5 mg/mL of concentration of enzyme solutions (pH 7, 0.05 M. sodium phosphate buffer) for 1 h. After enzyme immobilization, the nanogold membranes were washed thoroughly several times with sodium acetate buffer solution and were stored at 4° C. and were used further. The enzyme-nanogold membranes bioconjugate were used for biocatalytic measurements using carboxymethyl cellulose (CMC) as substrate. The membranes were reused for at the least 10 times. Prior to reuse membranes were washed with respective buffer solutions. Stability of enzyme endoglucanase bound to the nanogold membrane bioconjugates was checked in temperatures range 20-60° C. and in 2-10 pH range.

### EXAMPLE 6

This example illustrates immobilization of enzyme urease on the nanogold membrane. Immobilization of enzyme urease was done by immersing as-prepared nanogold membranes (10 mg) in 10 ml of 0.5 mg/mL of concentration of enzyme solutions (pH 7, 0.05 M sodium phosphate buffer) for 1 h. After enzyme immobilization, the nanogold membranes were washed thoroughly several times with sodium acetate buffer solution and stored at 4° C. and were further used. The enzyme-nanogold membranes bioconjugate were used for biocatalytic measurements using urea as the substrate. The membranes were reused for at least 10 times. Prior to reuse the membranes were washed with respective buffer solutions. Stability of enzyme urease bound to the nanogold membrane bioconjugates was checked in temperature range 20-60° C. and in 2-10 pH range.

### EXAMPLE 7

This example illustrates immobilization of enzyme trypsine on nanogold membrane. Immobilization of enzyme trypsine was done by immersing as-prepared nanogold membranes (10 mg) in 10 ml of 0.5 mg/mL of concentration of enzyme solutions (pH 7.5, 0.05 M sodium phosphate buffer) for 1 h. After enzyme immobilization, the nanogold membranes were washed thoroughly several times with sodium acetate buffer solution and were stored at 4° C. and were further used. The enzyme-nanogold membranes bioconjugate were used for biocatalytic measurements using N-benzoyl-L-arginine ethyl ester (BAEE) as substrate. The membranes were reused for at the least 10 times. Prior to reuse the membranes were washed with respective buffer solutions. Stability of enzyme trypsine bound to the nanogold membrane bioconjugates was checked in temperatures range 20-60° C. and in 2-10 pH range.

### EXAMPLE 8.

This example illustrates immobilization of enzyme cytochrome P450 on nanogold membrane. Immobilization of enzyme cytochrome P450 was done by immersing as-prepared nanogold membranes (10 mg) in 10 ml of crude extract of enzyme solutions in distilled water for 1 h. After enzyme immobilization, the nanogold membranes were washed thoroughly several times with distilled water and were stored at 4° C. and were used further. The enzyme-nanogold membranes bioconjugate were used for production of sophorolipids using unsaturated and saturated fatty acids such as arachidonic acid and oleic acid. The reactions were carried at 30° C. for 96 h under mild stirring conditions.

The present invention has been described in relation to certain preferred embodiments and several details have been set forth for purpose of illustration. It will however be apparent to those skilled in the art that the invention is susceptible to additional embodiments and that certain details described herein can be varied considerably without departing from the basic principles of the invention.

Advantages of the process claimed in the present invention are:

1. The main advantage of the invention is the use of biocompatible freestanding polymer membranes incorporated in situ with gold nanoparticles under static ambient experimental conditions on which enzymes can be immobilized.

2. Another advantage of the invention is that the enzyme nanogold membrane is reusable.

3. Another advantage of the invention is that the enzyme is easily separable from the reactions mixture and is reusable.

4. Another major advantage of the invention is that the enzyme bound to the nanogold membrane shows enhanced stability towards harsh temperature and pH conditions.

5. Requires less maneuvering and ambient experimental conditions.

6. Cost effective/Economical system for the industry.

7. As-prepared membranes and enzyme immobilised membranes are stable for long-term use.

8. More than one enzyme can be immobilized on nanogold membrane for multistep reactions.

9. The enzymes immobilized can withstand high temperature and pH conditions as compared to free enzyme in solution.

We claim:

1. A process for the preparation of immobilized enzymes on nano gold particles the method comprising mixing a dilute solution of chloroauric acid and a bis(2-(4-aminophenoxy)ethyl)ether (DAEE) in solvent and keeping the mixture in dark for a period of at least three hours to obtain a nanogold membrane at the organic/aqueous liquid-liquid interface, removing the membrane from the mixture, washing with double distilled water, drying at room temperature, contacting the gold nano membrane with a solution in buffer of enzyme to be immobilized, washing the membrane with buffer and drying to obtain the immobolized enzymes on nano gold membrane.

2. A process as claimed in claim 1 wherein the concentration of chloroauric acid is in the range of 10⁻² M to 10⁻⁴ M.

3. A process as claimed in claim 1 wherein the concentration of bis(2-(4-aminophenoxy)ethyl)ether (DAEE) is in the range of 10⁻² M to 10⁻⁴ M.

4. A process as claimed in claim 1 wherein the solvent for preparing solution of bis(2-(4-aminophenoxy)ethyl)ether (DAEE) is selected from the group consisting of chloroform, dichloromethane and ethyl acetate.

5. A process as claimed in claim 1 wherein the enzyme to be immobilized is selected from the group consisting of pepsin, fungal-protease, trypsine, endoglucanases and penicillin G acylase.

6. A process as claimed in claim 1 wherein the buffer used for the preparing solution of enzyme is selected from the group consisting of KCl-HCl, glycine-HCl, sodium citrate buffer, sodium phosphate buffer and glycine-NaOH buffer.

7. A process as claimed in claim 1 wherein the concentration of enzyme is in the range of 10⁻² M to 10⁻⁹ M.
